(19) United States
(12) Patent Application Publication
(10) Pub. No.: US 2007/0014746 A1
(43) Pub. Date: Jan. 18, 2007

(54) Hydrophobic emulsions containing polymers and process for obtaining same

(76) Inventors: Joaquin Bigorra Llosas, Sabadell (ES); Maria Escoda, Barcelona (ES); Otilia Par, Barcelona (ES); Heinz-Guenther Schulte, Kaarst (DE); Elisabet Graupera, Barcelona (ES)

Correspondence Address: COGNIS CORPORATION;PATENT DEPARTMENT, 300 BROOKSIDE AVENUE, AMBLER, PA, 19002, US

(21) Appl. No.: 11/432,592

(22) Filed: May 11, 2006

(30) Foreign Application Priority Data
May 11, 2005 (EP) 05010242.5

(51) Int. Cl. A61K 8/73; A61K 8/81; D06M 13/224

(52) U.S. Cl. 424/70.13; 424/70.16; 252/8.81

(57) ABSTRACT

An emulsion comprising polymers and method for making same is provided. The polymers consist of the following monomers: (a) 20 to 55% by weight acrylic and/or methacrylic acid; (b) 40 to 80% by weight C₁-C₄ alkyl (meth)acrylates; (c) 1 to 40% by weight of a maleic acid ester according to general formula (I)
R²OOC—CH═CH—COO(CH₂CH₂O)ₙR¹ (I)
wherein R¹ represents a linear or branched alkyl, aryl or alkaryl radical having 8 to 22 carbon atoms, R² represents hydrogen or (CH₂CH₂O)ₙR¹ and n represents a number between 2 and 150, (d) 0 to 15% by weight of aralphatic compounds having at least one double bond in the aliphatic side chain; and (e) 0 to 1% by weight polyethylenic unsaturated cross-linking monomers, wherein the amounts of components (a) to (e) total 100% by weight.

### CROSS-REFERENCE TO RELATED APPLICATIONS

This application claims priority under 35 U.S.C. § 119 from European Patent Application No. 05010242.5, filed May 11, 2005.

### FIELD OF THE INVENTION

The invention relates generally to emulsions comprising polymers, and more specifically, to emulsions comprising polymeric thickeners based on specific acrylates and maleic acid esters which exhibit their properties preferably under alkaline conditions, a process for making the polymers and various applications of said emulsions, for example, thickening paints.

### BACKGROUND INFORMATION

Polymeric thickeners belong to the state of the art for decades. Very well known are the so-called Carbopol types, polymers based on (meth)acrylic acid and/or their esters which are widely used in cosmetics. Thickening, however, is a phenomenon often associated with the formation and stabilization of mixed micelles and therefore, even for one skilled in the art, nearly impossible to predict. As a matter of fact, one thickener rather effective in one system or application can be worthless in another. This has lead to a huge number of polymeric thickening systems which have been obtained by a system of trial-and-error.

In the last years, certain polyurethane systems and especially copolymers of acrylic acid and maleic acid derivatives have been found rather interesting. For example, U.S. Pat. No. 3,657,175 and U.S. Pat. No. 4,351,754 (Rohm & Haas) disclose such products based on ethoxylated fatty alcohols. Copolymers of diisobutylene and partial esters of maleic acid and ethoxylated fatty alcohols and their use in cosmetics and detergents is known from EP 1191041 A1 and WO 01/096515 A1 (BASF). Similar copolymers based on olefins and esters of maleic anhydride and ethoxylated fatty alcohols are described in EP 0679669 A1 (Bayer); they serve, however, as pigment dispersants.

The major disadvantage of the products known either from patent literature or found in the market is their non satisfying thickening behavior, especially with respect to built up viscosity in paints and maintaining it even in case the products are stored at higher or lower temperatures. Therefore, the problem which underlies the present invention has been to find a new polymeric thickening system especially as auxiliary agent for paints and lacquers, which overcomes the disadvantages of the state of the art.

### SUMMARY OF THE INVENTION

Briefly described, according to an aspect of the invention an emulsion comprising polymers consists of the following monomers: (a) 20 to 55% by weight acrylic and/or methacrylic acid; (b) 40 to 80% by weight C₁-C₄ alkyl (meth)acrylates; (c) 1 to 40% by weight of a maleic acid ester according to general formula (I)
R²OOC—CH═CH—COO(CH₂CH₂O)ₙR¹ (I)
wherein R¹ represents a linear or branched alkyl, aryl or alkaryl radical having 8 to 22 carbon atoms, R² represents hydrogen or (CH₂CH₂O)ₙR¹ and n represents a number between 2 and 150, (d) 0 to 15% by weight of aralphatic compounds having at least one double bond in the aliphatic side chain; and (e) 0 to 1% by weight polyethylenic unsaturated cross-linking monomers, wherein the amounts of components (a) to (e) total 100% by weight.

According to another aspect of the invention, a process for making hydrophobic polymers includes copolymerizing the following compounds: 20 to 55% by weight acrylic and/or methacrylic acid; 40 to 80% by weight C₁-C₄ alkyl (meth)acrylates; 1 to 40% by weight of a maleic acid ester according to general formula (I)
R²OOC—CH═CH—COO(CH₂CH₂O)ₙR¹(I)
wherein R¹ represents a linear or branched alkyl, aryl or alkaryl radical having 8 to 22 carbon atoms, R² represents hydrogen or (CH₂CH₂O)ₙR¹ and n represents a number between 2 and 150; (d) 0 to 15% by weight of aralphatic compounds having at least one double bond in the aliphatic side chain; and, (e) 0 to 1% by weight polyethylenic unsaturated cross-linking monomers, wherein the amounts of components (a) to (e) total 100% by weight.

In another aspect of the invention, a monomeric emulsion consisting of: (a) 20 to 55% by weight acrylic and/or methacrylic acid; (b) 40 to 80% by weight C₁-C₄ alkyl (meth)acrylates; (c) 1 to 40% by weight of a maleic acid ester according to general formula (I)
R²OOC—CH═CH—COO(CH₂CH₂O)ₙR¹ (I)
wherein R¹ represents a linear or branched alkyl, aryl or alkaryl radical having 8 to 22 carbon atoms, R represents hydrogen or (CH₂CH₂O)ₙR¹ and n represents a number between 2 and 150, wherein the molar ratio of the monomers (a), (b) and (c) is about 1:0.5 to 1.5:0.1 to 0.5, and the amounts of components (a), (b) and (c) totals 100% by weight; and (d) an anionic emulsifier.

### DETAILED DESCRIPTION OF THE INVENTION

The invention includes hydrophobic alkali swellable emulsions (HASE), comprising polymers consisting of the following monomers:
(a) 20 to 55% b.w. acrylic and/or methacrylic acid,
(b) 40 to 80% b.w. C₁-C₄ alkyl (meth)acrylates,
(c) 1 to 40% b.w. of a maleic acid ester according to general formula (I)
   R²OOC—CH═CH—COO(CH₂CH₂O)ₙR¹ (I)
   in which R¹ stands for a linear or branched alkyl, aryl or alkaryl radical having 8 to 22 carbon atoms, R² stands for hydrogen or (CH₂CH₂O)ₙR¹ and n means a number between 2 and 150,
(d) 0 to 15% b.w. of aralphatic compounds having at least one double bond in the aliphatic side chain, and
(e) 0 to 1% b.w. polyethylenic unsaturated cross-linking monomers, calculated on the total composition of the polymers and under the condition, that compounds (a) to (e) add to 100% b.w.

Surprisingly it has been found that the emulsions according to the invention comprising the new polymers provide to paints and lacquers a high viscosity, which are also stable in case the products are stored over a longer period and at higher and lower temperature. For example, the new emulsions provide a viscosity to usual glossy and non-glossy paints based either on inorganic pigments or polymeric resins which is about double what can be achieved by using the same amount of polymeric thickeners according to the state of the art. In addition, the viscosity remains stable over a period of at least 8 weeks and at a storage temperature of 40 or 50° C. respectively, while under the same conditions products of the market lose about 50% of their viscosity.

Monomers

The polymers, which form the bases of the inventive emulsions, consist of at least three monomers:
(a) acrylic and/or methacrylic acid,
(b) C₁-C₄ alkyl (meth)acrylates, and
(c) maleic acid ester according to general formula (I).

It has, however, been found that with respect to component (a) mixtures of acrylic acid and methacrylic acid having a weight ratio of 10:90 to 90:10, preferably 40:60 to 60:40 show the best performance in thickening lacquers and paints. With respect to component (b) methyl esters of acrylic and/or methacrylic acid are the preferred ones. Among the maleic esters of general formula (I), which form component (c), those are preferred in which
R¹ represents a linear or branched alkyl radical having 12 to 18 carbon atoms and R² stands for hydrogen or a (CH₂CH₂O)ₙR¹ radical, and/or
n represents a number of 30 to 90.

The advantage of those esters is that they provide a very good temperature stability to the viscosity of the thickened products.

Optional Co-Monomers

In a preferred embodiment of the present invention the polymers may comprise additional co-monomers (component d), which represent aralphatic components, having at least one double bond in the aliphatic side chain. A typical example is styrene. The advantage of incorporating component (d) into the structure of the polymer is to provide to the products a high initial viscosity.

Cross-Linking Agents

According to a second preferred embodiment of the present invention said polymers may also contain cross-linking agents in order to improve the viscosity stability over time and under temperature stress. Examples of suitable cross-linkers are trimethylolpropane diallylether (TMPDAE), dipropylenglycol diacrylate (DPGDA) or allyl methacrylate.

Hydrophobic Alkali Swellable Emulsions (HASE)

In a third preferred embodiment of the present invention the polymers have the following composition:
(a) 25 to 50, preferably 30 to 40% b.w. acrylic and/or methacrylic acid,
(b) 50 to 70, preferably 60 to 65% b.w. C₁-C₄ alkyl (meth)acrylates,
(c) 2 to 30, preferably 5 to 25% b.w. of a maleic acid ester according to general formula (I),
(d) 0 to 10, preferably 1 to 5% b.w. of aralphatic compounds having at least one double bond in the aliphatic side chain, and
(e) 0.1 to 1% b.w. polyethylenic unsaturated cross-linking monomers,
   again calculated on the total composition of the polymer and always under the condition that components (a) to (e) add to 100% b.w. The emulsions shall comprise said polymeric thickeners in an amount of 1 to 50; preferably 2 to 40 and more preferably 5 to 30% b.w. (add water to give 100% b.w.).
   Process

Another object of the present invention is to provide a process for making hydrophobic polymers, which is characterized in that
(a) 20 to 55% b.w. acrylic and/or methacrylic acid,
(b) 40 to 80% b.w. C₁-C₄ alkyl (meth)acrylates,
(c) 1 to 40% b.w. of a maleic acid ester according to general formula (I)
   R²OOC—CH═CH—COO(CH₂CH₂O)ₙR¹ (I)
   in which R¹ stands for a linear or branched alkyl, aryl or alkaryl radical having 8 to 22 carbon atoms, R² stands for hydrogen or (CH₂CH₂O)ₙR¹ and n means a number between 2 and 150,
(d) 0 to 15% b.w. of aralphatic compounds having at least one double bond in the aliphatic side chain, and
(e) 0 to 1% b.w. polyethylenic unsaturated cross-linking monomers,
   calculated on the total composition of the polymers, under the condition, that compounds (a) to (e) add to 100% b.w. and are co-polymerized in known manner, which is illustrated, but not limited by the following procedure:
(i) In a first reactor a fatty alcohol polyglycolether corresponding to general formula (II)
   HO(CH₂CH₂O)ₙR¹ (II)
   in which R¹ and n have the same meanings as explained above, and maleic acid anhydride are placed in a molar ratio of 40:60 to 60:40. The mixture is heated for 2 to 5 h at 70 to 90° C. and the water of condensation separated off. Subsequently, the hemi ester thus obtained is cooled to 20° C.
(ii) In a second reactor water, a small amount of an anionic emulsifier like e.g. an alkyl sulphate and (meth)acrylic acid (component a), alkyl (meth)acrylate (component b) and the hemi ester obtained in step (i) (component c) are mixed to form a monomer emulsion. The molar ratio of the compounds (a):(b):(c) is about 1:0.5 to 1.5:0.1 to 0.5.
(iii) In a reactor water and again a small amount of said anionic emulsifier are placed and heated. Once the mixture reached 30° C. the reactor is purged with nitrogen and 10 to 20% by weight of the monomer emulsion prepared as described above, about 0.01 to 0.5% by weight iron sulphate, and about 0.01 to 1% by weight hydrogen peroxide are added. Finally, the mixture is treated with an aqueous solution of sulfoxylated sodium formaldehyde. Once the exothermic reaction has finalized, the remaining part of the monomer emulsion and another portion of the sulfoxylated sodium formaldehyde is added within about 1 to 2 hours, while the temperature is maintained at 40 to 45° C. After all compounds are added the temperature is raised to 50° C. and kept for about 1.5 hours. Then a third portion of the sulfoxylated sodium formaldehyde solution is added. After the mixture has cooled down to 20° C. the polymer is obtained in quantitative yield.

In case components (d) and (e) are also incorporated into the structure of the polymer, these compounds are added during step (ii). Finally, the hydrophobic polymers thus obtained are diluted in water so that an active matter content of 1 to 50, preferably 2 to 40 and more preferably 5 to 30% by weight is achieved.

### INDUSTRIAL APPLICATION

Another object of the present invention is directed to the use of said hydrophobic alkali swellable emulsions (HASE) as thickening agents, preferably by adding them to paints and lacquers. The final composition may comprise said emulsions in amounts of 0.1 to 2% by weight—calculated on active matter. In order to build up a high initial viscosity it has been found useful to combine the HASE with polysaccharides, preferably cellulose in ratios by weight of about 10:90 to 90:10 and more preferably 50:50 to 75:25.

It has also been found that the HASE provide a high and stable viscosity to products for other applications. Another object of the present invention is therefore the use of HASE as thickening agents for aqueous surface active compositions, like detergents, cosmetics and auxiliary agents for the treatment of textiles, fibers and leather.

### EXAMPLES

### Preparation Example 1

(i) In a first 1-I-laboratory reactor 500 g (0.24 mol) C₁₂-C₁₈ fatty alcohol+40 EO (Dehydol® PTA 40, Cognis Deutschland GmbH & Co KG) and 24.45 g (0.25 mol) maleic anhydride were placed. The mixture was heated for 3 h at 80° C. and the water of condensation separated off. Subsequently, the hemi ester thus obtained was cooled to 20° C.
(ii) In a second 5-I-laboratory reactor 2291 g water, 53 g (0.17 mol) lauryl sulphate, 1197 g (11.97 mol) ethyl acrylate, 598 g (6.95 mol) methacrylic acid and 199 g (0.095 mol) of the hemi ester obtained in step (i) were mixed to form a monomer emulsion.
(iii) In a third 5-I-laboratory reactor 1820 g water and 25 g (0.08 mol) lauryl sulphate were placed and heated. Once the mixture reached 30° C. the reactor was purged with nitrogen and 10% by weight of the monomer emulsion prepared as described above, 0.27 g iron sulphate, 19 g water, and 6.7 g hydrogen peroxide were added. Finally, the mixture was treated with a 10 Vol.-% of a solution of 2.1 g (0.018mol) Rongalita® CF (sulfoxylated sodium formaldehyde, SSF) in 385 ml of water. Once the exothermic reaction has finalized, the remaining part of the monomer emulsion and the rest of the SSF solution was added within 1.75 hours, while the temperature was maintained at 40 to 45° C. After all compounds had been added the temperature was raised to 50° C. and kept for 1.5 hours. Then a third portion of 1.4 g (0.012 mol) SSF in 245 g water was added. After the mixture has cooled down to 20° C. the polymer was obtained in quantitative yield.

### Preparation Example 2

(i) In a first 1-I-laboratory reactor 500 g (0.24 mol) C₁₂-C₁₈ fatty alcohol+40 EO (Dehydol® PTA-40, Cognis Deutschland GmbH & Co KG) and 24.45 g (0.25 mol) maleic anhydride were placed. The mixture was heated for 3 h at 80° C. Subsequently, the hemi ester thus obtained was cooled to 20° C.
(ii) In a second 5-I-laboratory reactor 1722.4 g water, 40.0 g (0.13 mol) lauryl sulphate, 900 g (9.00 mol) ethyl acrylate, 525 g (6.10 mol) methacrylic acid, 150 g (0.071 mol) of the hemi ester obtained in step (i) and 5.0 g (0.027 mol) of trimethylolpropane diallylether were mixed to form a monomer emulsion.
(iii) In a third 5-I-laboratory reactor 1500 g water and 18.6 g (0.06 mol) lauryl sulphate were placed and heated: Once the mixture reached 30° C. the reactor was purged with nitrogen and 10% by weight of the monomer emulsion prepared as described above, 0.20 g Iron sulphate, 19.8 g water, and 5.0 g hydrogen peroxide were added. Finally, the mixture was treated with a 10 Vol.-% of a solution of 1.56 g (0.013 mol) Rongalita® CF in 287 ml of water. Once the exothermic reaction has finalized, the remaining part of the monomer emulsion and the rest of the SSF solution was added within 1.75 hours, while the temperature was maintained at 40 to 45° C. After all compounds had been added the temperature was raised to 50° C. and kept for 1.5 hours. Then a third portion of 1.042 g (0.009 mol) SSF in 182.4 g water was added. After the mixture has cooled down to 20° C. the polymer was obtained in quantitative yield.

### Formulation Example F3

The following table 1 discloses the composition of a paint comprising the new thickening emulsion, more particularly, the term thickener in the composition means 0.2% by weight of the polymer emulsion obtained according to example 1+0.1% by weight cellulose. The solids content of the paint has been 59.0% by weight, the relation between pigments and resins was 5.5:1, and the pigment volume concentration (PVC) resulted to 67.5% by weight.

TABLE 1
Paint composition (all concentrations % b.w.)

| | |
|---|---|
| Calcium carbonate | 38.00 |
| Titan dioxide | 12.00 |
| Other pigments | 18.00 |
| Hydropalat ® 5040 Polyacrylate dispersant | 0.20 |
| Thickening agent | 0.20 |
| Foamaster ® 50 Antifoaming agent | 0.15 |
| based on mineral oil | |
| Sodium hexametaphosphate | 0.10 |
| Ammonia (25% b.v.) | 0.10 |
| Biocides | 0.10 |
| Water | add to 100% |

### Application Example A4

In the following the viscosity of flat paints based on and acryl-styrene resins (Acronet® 2380, Acronal® 290 D, BASF) has been determined by adding 0.3 b.w. of different thickening agents. The tests have been carried out according to the Brookfield method (spindle 4, 10 rpm, 20° C.) and are reflected in Table 2:

TABLE 2
Viscosity of flat paints (mPas)

| | Acronet ® 2380 | Acronal ® 290 D |
|---|---|---|
| Rheolate ® 420¹ | 15.420 | 13.900 |
| Acrysol ® TT 935¹ | 26.300 | 17.820 |
| HASE 40² | 42.900 | 24.700 |
| HASE 45² | 36.130 | |

¹Rheolate ® and Acrysol ® are polymeric thickeners of Rohm & Haas and Elementis
²HASE 40 is a thickener according to the invention prepared according to Example 1 based on tallow fatty alcohol + 40EO while HASE 45 is based on behenyl alcohol + 20EO, both polymers combined with cellulose in a weight ratio of 2:1.

What is claimed is:

1. An emulsion comprising polymers consisting of the following monomers:
(a) 20 to 55% by weight acrylic and/or methacrylic acid;
(b) 40 to 80% by weight C₁-C₄ alkyl (meth)acrylates;
(c) 1 to 40% by weight of a maleic acid ester according to general formula (I)
R²OOC—CH═CH—COO(CH₂CH₂O)ₙR¹ (I)
wherein R¹ represents a linear or branched alkyl, aryl or alkaryl radical having 8 to 22 carbon atoms, R² represents hydrogen or (CH₂CH₂O)ₙR¹ and n represents a number between 2 and 150,
(d) 0 to 15% by weight of aralphatic compounds having at least one double bond in the aliphatic side chain; and
(e) 0 to 1% by weight polyethylenic unsaturated cross-linking monomers,
wherein the amounts of components (a) to (e) total 100% by weight.

2. The emulsion according to claim 1, wherein component (c) represents a maleic acid ester of formula (I) in which R¹ is a linear or branched alkyl radical having 12 to 18 carbon atoms and R² is hydrogen or a (CH₂CH₂O)ₙR¹ radical.

3. The emulsion according to claim 1, wherein component (c) represents a maleic acid ester of formula (I) in which n represents a number of 30 to 90.

4. The emulsion according to claim 1, wherein component (c) is styrene.

5. The emulsion according to claim 1, wherein component (d) is selected from the group consisting of: trimethylolpropane diallylether, dipropylenglycol diacrylate and allyl methacrylate.

6. The emulsion according to claim 1, wherein the polymers are present in the emulsion in an amount of 1 to 50% by weight.

7. A process for making hydrophobic polymers, comprising the step of copolymerizing the following compounds:
(a) 20 to 55% by weight acrylic and/or methacrylic acid;
(b) 40 to 80% by weight C₁-C₄ alkyl (meth)acrylates;
(c) 1 to 40% by weight of a maleic acid ester according to general formula (I)
R²OOC—CH═CH—COO(CH₂CH₂O)ₙR¹ (I)
wherein R¹ represents a linear or branched alkyl, aryl or alkaryl radical having 8 to 22 carbon atoms, R² represents hydrogen or (CH₂CH₂O)ₙR¹ and n represents a number between 2 and 150;
(d) 0 to 15% by weight of aralphatic compounds having at least one double bond in the aliphatic side chain; and,
(e) 0 to 1% by weight polyethylenic unsaturated cross-linking monomers,
wherein the amounts of components (a) to (e) total 100% by weight.

8. The process for making hydrophobic polymers according to claim 7, wherein the hydrophobic polymers are diluted in water so that an active matter content of 1 to 50% by weight is achieved.

9. The emulsion according to claim 1, incorporated into a composition as a thickening agent.

10. The emulsion according to claim 1, incorporated into a paint or lacquer composition.

11. The emulsion according to claim 9, wherein the emulsion is present in the composition in an amount of 0.1 to 2% by weight based on active matter.

12. The emulsion according to claim 9, wherein the composition includes polysaccharides.

13. The emulsion according to claim 10, wherein the emulsion is present in the composition in an amount of 0.1 to 2% by weight based on active matter.

14. The emulsion according to claim 10, wherein the composition includes polysaccharides.

15. The emulsion according to claim 1, incorporated into an aqueous surface active composition as a thickening agent.

16. The emulsion according to claim 15, wherein the aqueous surface active composition is a detergent, cosmetic, or auxiliary agent for the treatment of textiles.

17. The emulsion according to claim 1, further comprising an anionic emulsifier.

18. The emulsion according to claim 17, wherein the anionic emulsifier is an alkyl sulphate.

19. A monomeric emulsion consisting of:
(a) 20 to 55% by weight acrylic and/or methacrylic acid;
(b) 40 to 80% by weight C₁-C₄ alkyl (meth)acrylates;
(c) 1 to 40% by weight of a maleic acid ester according to general formula (I)
R²OOC—CH═CH—COO(CH₂CH₂O)ₙR¹ (I)
wherein R¹ represents a linear or branched alkyl, aryl or alkaryl radical having 8 to 22 carbon atoms, R² represents hydrogen or (CH₂CH₂O)ₙR¹ and n represents a number between 2 and 150, wherein the molar ratio of the monomers (a), (b) and (c) is about 1:0.5 to 1.5:0.1 to 0.5, and the amounts of components (a), (b) and (c) totals 100% by weight; and
(d) an anionic emulsifier.

20. The monomeric emulsion according to claim 19, wherein the anionic emulsifier is an alkyl sulphate.
